(19) United States
(12) Patent Application Publication
(10) Pub. No.: US 2018/0177167 A1
(43) Pub. Date: Jun. 28, 2018

(54) RAISED GARDEN BUTTERFLY CONSERVATORY

(71) Applicant: VEGHERB, LLC, Cary, NC (US)

(72) Inventor: Anthony Topping, Cary, NC (US)

(21) Appl. No.: 15/851,911

(22) Filed: Dec. 22, 2017

Related U.S. Application Data
(60) Provisional application No. 62/439,662, filed on Dec. 28, 2016.

(51) Int. Cl. A01K 67/033; A01G 9/28; A01K 1/00; A01G 22/60; A01G 9/16; A01G 9/14

(52) U.S. Cl. CPC A01K 67/033; A01G 9/28; A01K 1/0058; A01K 2227/706; A01G 9/16; A01G 9/1407; A01G 22/60

(57) ABSTRACT

A reconfigurable butterfly conservatory includes a raised garden base that defines an inner garden area. The raised garden base includes holes for receiving complementary upright poles. The conservatory further includes a canopy structure that includes connectors for receiving the complementary upright poles, wherein the canopy structure includes an air vent and one or more first windows that can be opened and closed. The air vent includes a mesh layer that promotes chrysalis formation. The conservatory further includes a plurality of side walls that are configured for coupling to at least the canopy roof structure for suspending the plurality of side walls such that bottom edges of the side walls are positioned adjacent to the raised garden base to define a hollow interior space that includes the inner garden area. At least one side wall is formed at least substantially of a mesh material.

### CROSS REFERENCE TO RELATED APPLICATION

The present application claims priority to U.S. patent application Ser. No. 62/439,662, filed Dec. 28, 2016, which is hereby incorporated by reference in its entirety.

### TECHNICAL FIELD

The present application relates generally to a multi-position structure and more specifically, to a raised garden butterfly conservatory that serves a migration station to provide a secure area for butterflies to undergo their life cycle and multiply in count.

### BACKGROUND

One of the more fascinating things to observe in nature is the full life cycle of a butterfly. A butterfly's life cycle is defined by four unique stages of life and all butterflies undergo a complete metamorphosis. More specifically, in order to grow into an adult, butterflies go through four stages, namely, a first stage: egg; a second stage: larva; a third stage: pupa; and a fourth stage: adult. Each of these stages has a different objective and depending upon the type of butterfly, the life cycle of a butterfly may take anywhere from one month to a whole year. As previously mentioned, the first stage is the egg stage in which a butterfly starts life as a very small, round, oval or cylindrical egg. The shape of the egg depends of the type of butterfly that laid the egg and generally are round or oval shape. The second stage is the larva stage, which can also be referred to as the caterpillar stage, since butterfly larvae are actually what we call caterpillars. Caterpillars do not stay in this stage for very long and mostly, in this stage all they do is eat in order to keep growing and prepare for the next stage. The eggs are typically laid on the leaves of plants that the caterpillar will eat. Once the egg hatches, the caterpillar starts eating the leaves of the plant immediately and since initially, the caterpillar is very small, the caterpillar does not travel far and thus, the egg must be laid on the type of leaf that the caterpillar likes to eat. The third stage is a pupa (chrysalis) stage in which a caterpillar becomes a chrysalis. As soon as a caterpillar is done growing and they have reached their full length/weight, they form themselves into a pupa, also known as a chrysalis. Inside of the pupa, the caterpillar is rapidly changing and more particularly, within the chrysalis the old body parts of the caterpillar are undergoing a remarkable transformation, called ‘metamorphosis,’ in which the old parts become the beautiful parts that make up the butterfly that will emerge from the chrysalis. Tissue, limbs and organs of a caterpillar have all been changed by the time the pupa is finished, and is now ready for the final stage of a butterfly's life. The fourth and final stage is the adult butterfly stage. Finally, when the caterpillar has done all of its forming and changing inside the pupa, an adult butterfly emerges from the chrysalis. The butterfly will initially rest and then prepares the wings for flight. The objective of this fourth stage is to find a mate in order to reproduce and when the female butterfly lays eggs on leaves, this entire cycle will start over.

It will be appreciated that the entire life cycle of the butterfly is very fragile and at any one point in the stage, many obstacles must be overcome. For example, there are environmental conditions and predators that challenge the survival of the caterpillar/butterfly. The eggs can be eaten or can be dislodged from the leaf. In addition, pesticides meant to kill other insects kills the butterflies and also, the specific food source for butterflies can be harmed by herbicides, farming and urbanization. Butterflies are also threatened by global warming and increased storms are taking a toll on butterflies.

One of the more well-known butterflies is a monarch butterfly; however, monarch butterflies like others are facing an increasingly threatening environment and the recorded number of butterflies continues to drop.

There is therefore a need for a product that not only educates children and adults as to the fascinating life cycle of the butterfly but also provides a means to assist in butterfly population regrowth. In particular, this is a need for an easy to use and easy to assemble butterfly conservatory product not only provides a protected shelter for butterflies but also provides a means for initially growing the food source of the butterfly and associated caterpillar. The present invention provides such a product that natures and promotes population regrowth.

### SUMMARY

A reconfigurable butterfly conservatory includes a raised garden base that defines an inner garden area. The raised garden base includes holes for receiving complementary upright poles. The conservatory further includes a canopy structure that includes connectors for receiving the complementary upright poles, wherein the canopy structure includes an air vent and one or more first windows that can be opened and closed. The air vent includes a mesh layer that promotes chrysalis formation. The conservatory further includes a plurality of side walls that are configured for coupling to at least the canopy roof structure for suspending the plurality of side walls such that bottom edges of the side walls are positioned adjacent to the raised garden base to define a hollow interior space that includes the inner garden area. At least one side wall is formed at least substantially of a mesh material.

### BRIEF DESCRIPTION OF THE DRAWING FIGURES

FIG. 1 is an exploded view showing the components of a conservatory in accordance with at least one embodiment;

FIG. 2 is a perspective view of the raised garden base in accordance with at least one embodiment;

FIG. 3A is a perspective view of the canopy structure in accordance with at least one embodiment;

FIG. 3B is a top plan view of the canopy structure in accordance with at least one embodiment;

FIG. 4A is bottom and side perspective view of a hub of the canopy structure in accordance with at least one embodiment;

FIG. 4B is a bottom plan view of the hub of the canopy structure without the pole members (ribs) in accordance with at least one embodiment;

FIG. 4C is a top plan view of the hub of the canopy structure in accordance with at least one embodiment;

FIG. 5A is a perspective view of a pole connector of the conservatory in accordance with at least one embodiment;

FIG. 5B shows a coupling member mated to the pole connector in accordance with at least one embodiment;

FIG. 5C is a cross-sectional view showing the coupling member mated to the pole connector in accordance with at least one embodiment;

FIG. 6 is a bottom perspective view of the canopy structure in a relaxed (non-tensioned) state in accordance with at least one embodiment;

FIG. 7 is a bottom perspective view of the canopy structure in a tensioned state which represents an in-use state of the canopy structure in accordance with at least one embodiment;

FIG. 8A is a perspective view of the conservatory in a raised frame configuration featuring a canopy structure, side wall assembly, and raised garden base in accordance with at least one embodiment;

FIG. 8B is a perspective view of the conservatory in a raised frame configuration featuring a canopy structure, side wall assembly, and raised garden base in accordance with at least other embodiment;

FIG. 8C is a diagram of the attachment of the side walls of the side wall assembly to one another in accordance with at least one embodiment;

FIG. 8D is a diagram of the attachment of the side walls to the canopy structure in accordance with at least one embodiment;

FIG. 9 is a perspective view of the canopy structure in a cold frame configuration in accordance with at least one embodiment;

FIG. 10A is a side view of the canopy structure and the raised garden base in a cold frame configuration in accordance with at least one embodiment; and

FIG. 10B is a top view of the canopy structure and the raised garden base in a cold frame configuration in accordance with at least one embodiment.

### DETAILED DESCRIPTION OF CERTAIN EMBODIMENTS OF THE DISCLOSURE

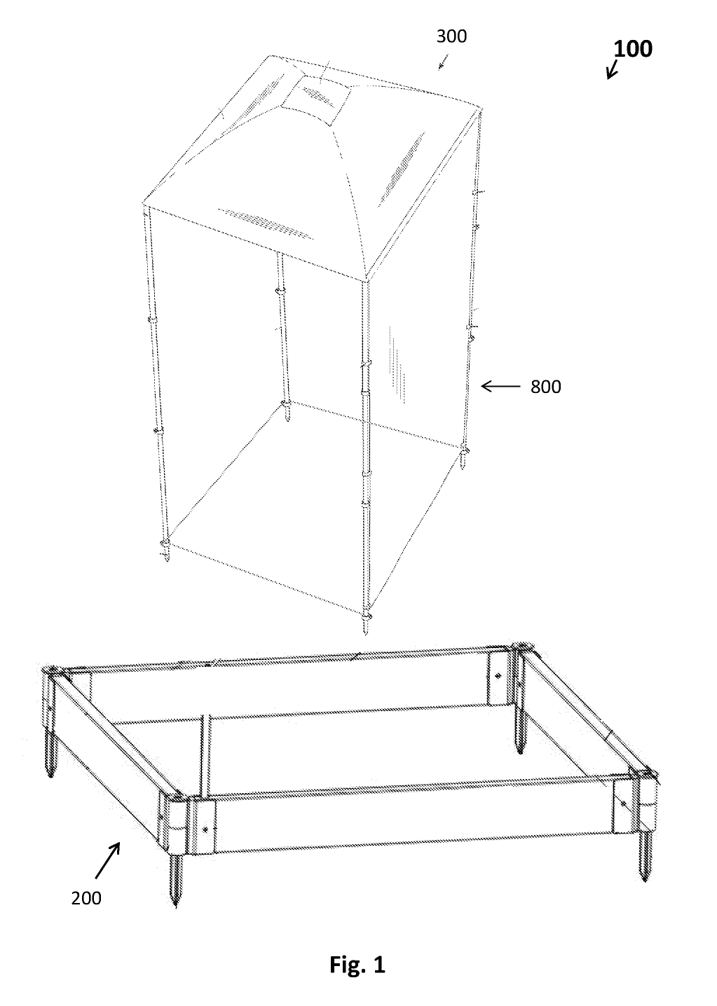

In accordance with the present invention, a raised garden butterfly conservatory (“conservatory”) 100 is provided and is shown in the illustrated figures (see, e.g., FIG. 1). As described herein, the raised garden butterfly conservatory 100 has a number of different operating positions/states. More specifically, the operating states include but are not limited to: (1) a cold frame configuration that protects seedlings and small plants during cooler weather; and (2) a raised frame configuration that can act as a standup greenhouse, an animal barrier for any garden that a user wants to grow; and/or a butterfly house. In one or more embodiments described herein, while the conservatory 100 is intended for use as a butterfly house, it also provides a structure that facilitates the growing of plants, including the plants that caterpillars eat to become butterflies. For example, monarch butterflies eat milkweed plants and therefore, to encourage monarch butterfly survival, the conservatory 100 provides an environment in which milkweed can grow and ultimately, butterfly eggs can be safely protected to allow them to hatch into caterpillars to begin the cycle discussed hereinbefore.

The conservatory 100 is typically provided in the form of a kit that is formed of a number of assemblies. In particular, as shown in FIG. 1, the conservatory 100 can be formed of a raised garden base 200; a canopy structure 300; and a side wall assembly 800 that can be coupled to the raised garden base 200 and the canopy structure 300 to provide wall structures that extend therebetween. In one or more embodiments, the conservatory 100 can have length and width dimensions of 4 feet and 4 feet, respectively (4×4). In at least one embodiment, the dimensions of the conservatory is 5×7 feet. In one or more embodiments (e.g., hexagonal embodiment), the dimensions of the conservatory is 7×8 feet. However, it should be understood that the width and length dimensions of the conservatory 100 can vary and can be designed to fit any number of settings including a small balcony of an apartment. Further, the height of the conservatory 100 can also vary, as the legs (poles) of the side wall assembly 800 can vary in length.

Raised Garden Base

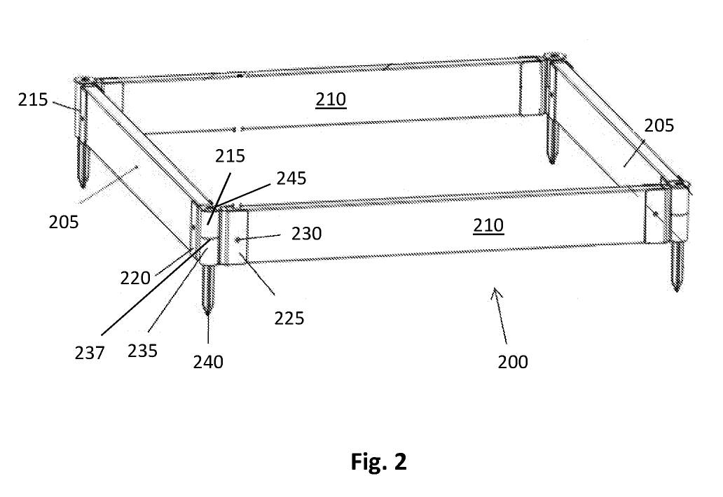

An exemplary raised garden base 200, in accordance with one or more embodiments, is shown at FIG. 2. The raised garden base 200 is the base structure of the conservatory 100 that is constructed and sits on a ground surface, e.g., a garden area in a yard. The raised garden base 200 is configured to define a raised enclosed border that rests on the ground surface. In general, the raised garden base 200 is formed of a plurality of interlocking support members coupled to one another, typically in the corners. The raised garden base 200 can also be described as being a raised border base.

In one exemplary embodiment, a number of timbers or the like (e.g., fabricated planks) are joined together by support brackets (coupling members), substantially as described in U.S. Pat. No. 6,202,367, the disclosure of which is hereby incorporated by reference as if set forth in its entirety herein. In one or more implementations, the timbers can be composed of wood grain timbers that are not susceptible to rotting, warping, splitting, or fading, or to infestations by termites, ants, or the like. The timbers can also be made from recycled plastic and/or natural fibers. The raised garden base 200 can be of a modular type that are constructed of a number of different components that mate together to form a border. Typically, the raised border systems include a number of different joints or interfaces, such as corners, between side walls, etc.

With reference to FIG. 2, in one or more embodiments, the raised base 200 includes a pair of end planks (timbers) 205 and a pair of side planks (timbers) 210. When the raised base 200 is square shaped, the end planks 205 and side planks 210 can be the same (i.e., same lengths). The end and side planks 205, 210 can be formed of any number of different materials, including but not limited to wood, plastics, metal, masonry, composite wood grain, etc.

The raised base 200 has a set of connectors 215 which each has first and second receiving portions 220, 225 for receiving respective ends of one end plank and one side plank. The first and second receiving portions 220, 225 can be formed at a right angle as shown. Each of the first and second receiving portion 220, 225 can thus include a receiving space or slot into which the respective end is inserted. A fastener 230 can be used to securely anchor the end of the plank within the slot. For example, a screw or nail can be used. When the support connector 215 is a corner bracket, the side and end plank members 205, 210 are formed at a substantially right angle. In at least one embodiment, the end and side planks 205, 210 can be stackable, such that height of the raised bed 200 can be increased. In these embodiments, additional support connectors 215 can be used to accommodate the stacked planks 205, 210.

Each connector 215 has a main portion 235 formed between the first and second receiving portions 220, 225. The main portion 235 has a first bore 237 formed therein along a first axis. An anchor member 240, such as a stake, can extend downwardly from the main portion 235 for securely anchoring one corner of the raised base 200 to a ground surface. In addition, the main portion 235 includes a second bore 245 that is open at the top and receives the bottom end of a leg of the side wall assembly or a leg of the canopy as discussed in further detail below. The first and second bores are preferably axially aligned. Any number of different techniques, including locking components, can be used to securely lock the leg of the side wall assembly in place within the main portion 235. For example, a friction fit can be used or a locking mechanism can be used to lock the leg in place. The insertion of the legs into the corner connectors 215, thereby couples the side wall assembly to the raised base 200.

As mentioned, the combination of the raised base 200, canopy structure 300, and the side wall assembly 800 (collectively, the conservatory 100) can define a sand box structure, a butterfly enclosure, a garden bed, etc. The canopy structure 300 is elevated above the raised base 200 at a set distance and thus shelters the area defined internally within the raised base 200. The anchoring of the raised base 200 to the ground via anchor member 240 helps to prevent the conservatory 100 from falling over due to storms or high winds.

With continued reference to FIG. 2, the connectors 215 can be formed of any number of different materials including plastics and in particular, the connectors 215 can be a single molded piece of plastic (e.g., injection molded piece). It will be appreciated that any number of other connectors can be used to couple together the side and end plank members 205, 210 to form the raised bed 200, so long as the connectors also include an opening or the like for receiving and securely holding a vertical leg for attachment to the side wall assembly 800.

Exemplary raised garden bases 200 are disclosed in U.S. Pat. No. 8,955,251, which is hereby incorporated by reference in its entirety.

The raised garden base 200 thus provides a border around a garden area.

Canopy Structure

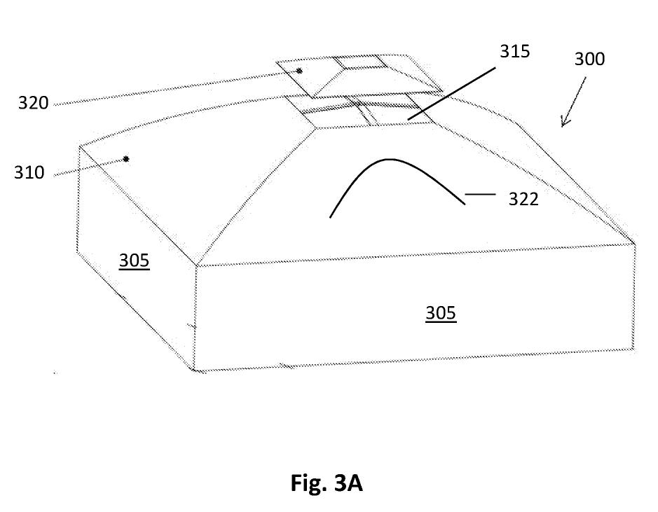

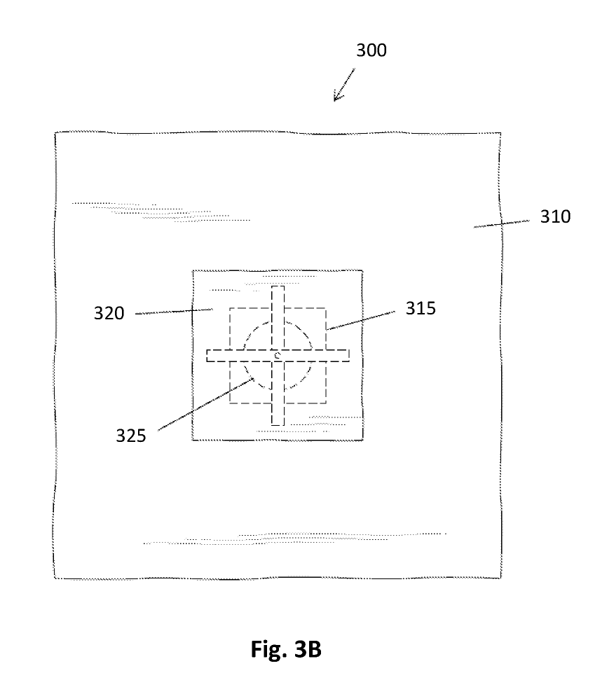

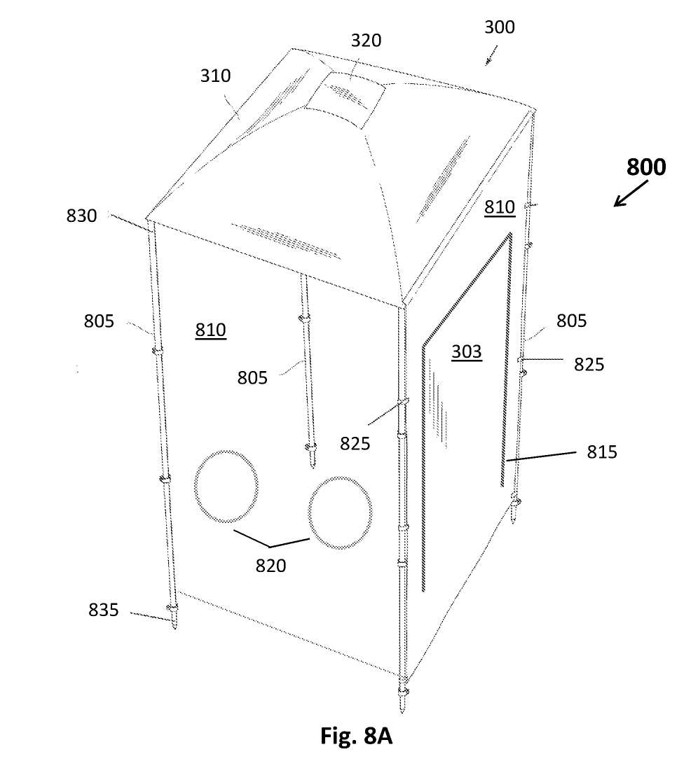

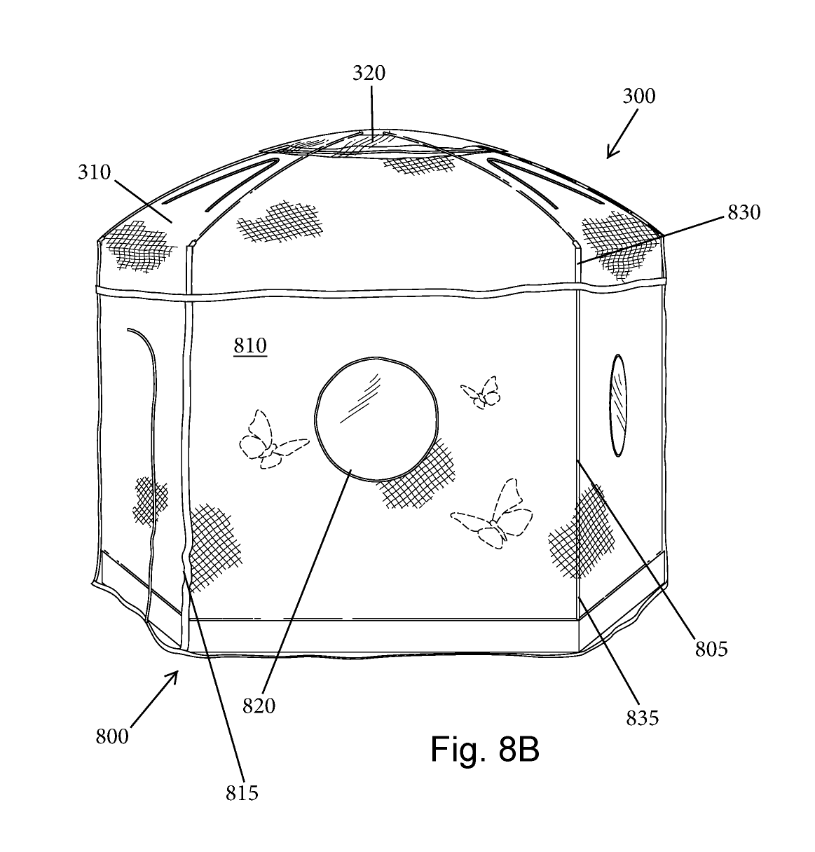

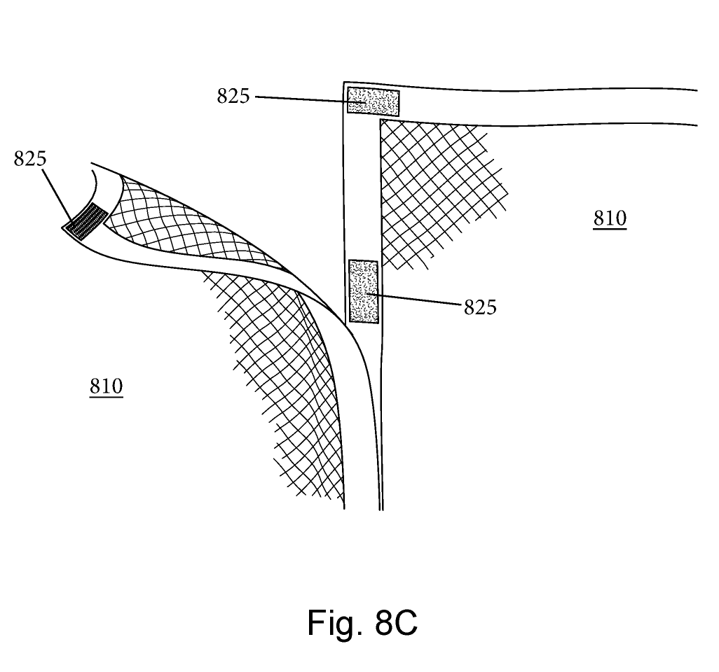

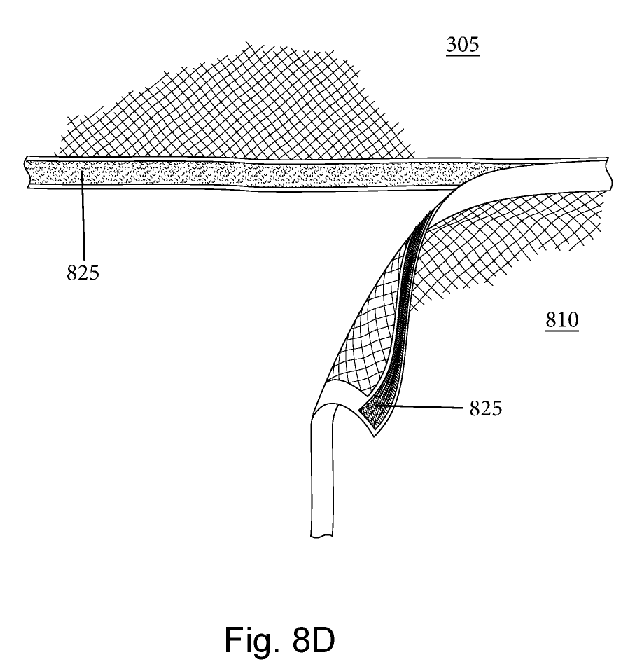

An exemplary collapsible canopy structure 300, in accordance with one or more embodiments, is shown at FIGS. 3A and 3B. The canopy structure 300 offers protection for butterflies/caterpillars in the conservatory 100 from severe elements and rain storms and high winds. It will be appreciated that any number of different canopy structures can be used with the conservatory 100 so long as the canopy structure is configured to provide the features described herein. The canopy structure 300 can be formed of any number of different types of material, including but not limited to one or more plastics, polyvinyl chloride (PVC), a nylon material, a canvas material, other natural or synthetic materials, or a combination of materials. As will be described below, the canopy structure 300 can be securely coupled to the raised garden base 200 directly, or indirectly via coupling with the side wall assembly 800. The indirect coupling of the canopy structure 300 with the base 200 via the side wall assembly 800 is such that the side wall assembly 800 extends between the canopy 300 and the base 200 so as to create and define a covered space (see FIG. 1). Also, see FIG. 8B for a similar structure.

FIG. 3A is a side perspective view of an exemplary canopy collapsible structure 300 in accordance with at least one embodiment. As shown in FIGS. 3A, the canopy 300 can include side panels 305, a first main canopy 310, a vent 315, and a secondary canopy 320. The first main canopy 310 and the secondary canopy 320 have complementary shapes as discussed herein. In the illustrated embodiment, the first main canopy 310 has a square shape and the secondary canopy 320 also has a square shape.

As shown in FIG. 3A, in one or more embodiments the main canopy 310 can also include one or more windows 322. The windows 322 can be selectively opened via one or more fasteners, such as a zipper. The windows 322 provide a user with easy access to the interior of the conservatory 100 for maintenance of the plants and/or caterpillars. In at least one embodiment, the window 322 can feature double zippers, which allow a user to access the window 322 from inside and outside of the conservatory 100. The one or more windows 322 can be used to allow butterflies to enter or exit the conservatory 100. FIG. 3A shows the window 322 as a v-shaped window, but it should be understood that the window 322 can be made in any number of different shapes.

It will be appreciated that the first main canopy 310 and the secondary canopy 320 can have any number of different shapes and are not limited to the square shapes shown in the figures. The first main canopy 310 is larger than the secondary canopy 320.

FIG. 3B shows a top plan view of the exemplary collapsible canopy structure 300 in accordance with at least one embodiment. It will be appreciated that canopy structure 300 is show in different configurations (e.g., shapes and sizes) herein and will be appreciated that it can be formed to have different shapes and sizes. As mentioned above, the first main canopy 310 includes a vent 315 formed centrally therein. The vent 315 in the illustrated embodiment has a square shape. In one or more implementations, the vent 315 can be a mesh material. In this embodiment, the mesh vent 315 can operate as a beacon to attract butterflies to conservatory 100, as it allows for flower fragrances to exit the conservatory 100. The secondary canopy 320 overlies the vent 315 and is securely attached to the first main canopy 310 at select locations to allow air to be vented through the vent 315. The vent 315 can also protects against an unintended inversion of the collapsible shelter 100 and/or damage occurring due to wind blowing underneath the first main canopy 310. The secondary canopy 320 can thus be considered to be a cap or dome over the vent 315.

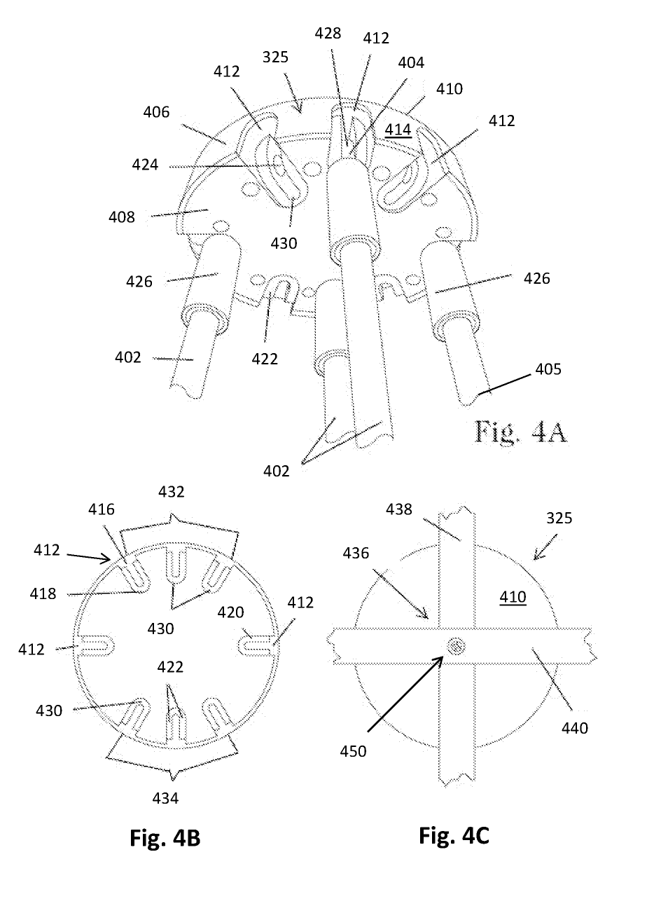

It will be appreciated that the secondary canopy 320 is not sealed along all the entire lengths of its edges since this would prevent venting from occurring. Instead, there are sections of the secondary canopy 320 that are not attached (sealed) to the underlying first main canopy 310 and the gaps between the points of attachment define air flow paths for venting the air. The secondary canopy 320 covers the vent (opening) 315 so as to not allow any of the elements (e.g., rain) to pass therethrough but allows airflow. The canopy structure 300 can also include a central hub 325, which is shown in greater detail in FIGS. 4A-C.

FIGS. 4A-C show various views of the center hub framework. The framework includes a central hub 325 and a plurality of ribs 402 that are pivotably coupled to the central hub 325 at first ends 404 thereof and to the first main canopy 310 at second ends 405 thereof. The central hub 325 is defined by a body 406 that has a first surface or face 408 and an opposing second surface of face 410. The first face 408 faces upward in a direction toward the secondary canopy 320, while the second face 410 faces downward toward the ground surface.

The central hub 325 is shaped and sized to be received within the vent 315 formed in the first main canopy 310. In the illustrated embodiment, the central hub 325 has a disk shape (circular shape). The first face 408 includes a plurality of locking slots 412 formed therein for allowing the pivoting of the ribs 402 relative to the central hub 325. The locking slots 412 are integrally formed in the central hub 325 and are open along the first face 408 and along a peripheral side surface 414 that extends between the first face 408 and the second face 410. When the central hub 325 is in the form of a disk, the peripheral side surface 325 has an annular shape. For each rib 402, there is a corresponding locking slot 412 to receive the rib 402 and allow pivoting thereof.

In the exemplary illustrated embodiment, the locking slot 412 has a U-shape in that each locking slot 412 has an open end 416 and a closed end 418. The closed end 418 can be a curved end as shown. The locking slot 412 does not extend completely to the second face 410 and thus, the locking slot 412 defines a stop 430 which limits the degree of travel (pivoting) of the rib 402 relative to the central hub 325. This prevents over rotation of the rib 325 relative to the central hub 325. The stop 430 can thus be thought of as being a bearing surface. This ensures that each of the ribs is in a desired position when the ribs are fully extended and also prevents against an unintended inversion of the canopy.

As shown in FIGS. 4A and 4B, each locking slot 412 has a structure that allows for the first end 404 of the rib 402 to be securely attached to the central hub 325 and more specifically, allows for the first end 404 to pivot relative to the central hub 325. In one exemplary construction, an axle associated with the first end 404 of the rib 402 can be received within openings or slots formed in opposing side walls of the locking slot 412 to allow for the pivoting of the rib 402 relative to the central hub 325. For example, within the slot 412, a connector 420 can be disposed and as shown in FIG. 4A and 4B, the connector 420 can have a U-shape complementary to the U-shape slot. The opposing side walls 422 of the connector 420 can have an opening (hole) 424 which can receive an axle (not shown). The opposing ends of the axle are received in the openings 424.

Proximate the first end 404, the rib 402 can have a sleeve 426 disposed around the rib 402. The sleeve 426 can thus be in the form of a hollow member that receives the distal end of the rib 402 resulting in the sleeve 426 being securely attached to the distal end of the rib 402. The sleeve 426 can be formed of any number of different materials, such as metal or a rigid plastic. The fit between the sleeve 426 and the rib 402 can be of a friction nature (friction fit) and/or an adhesive agent can be used to securely attach the rib 402 to the inside of the sleeve 426.

The sleeve 426 is formed such that at a distal end thereof contains an integral finger or extension 428. The finger 428 can contain a bore that extends transversely therethrough to allow the axle to pass through the finger, thereby coupling the sleeve 426 to the hub 325 in a pivotable manner. The size of the finger 428 is selected so that it can be received between the two side walls 422 (however, preferably there is not excessive space between the finger 428 and the side walls 422 so as to prevent excessive lateral movement of the finger 428).

Thus, the sleeve 426 is sized so that at least a portion thereof can be received within the locking slot 412 and can pivot therein; however, when the rib 402 is pivoted into a fully extended position, the sleeve 426 contacts (bears against) the stop 430 and this limits the degree of travel of the rib 402. In other words, the rib 402 can only be pivoted in a direction toward the central hub 325 for extending the rib 402 until the sleeve 426 contacts the stop 430.

It will be appreciated that the central hub can be constructed so as to not include the connectors 420 and instead have other structures that allow for pivoting of the ribs within the locking slots. For example, instead of an axle being used and extending through a hole in the finger 428, the first end 404 of the rib 402 can include a pair of posts that extend radially outward in opposite directions in a co-axial manner. These posts are received in openings (slots) formed in the locking slot to allow for the pivoting of the rib relative to the respective locking slot. In this case, the separate axle and connector 420 are eliminated or at least the axle is eliminated.

As shown in FIG. 4B, the locking slots 412 can be formed and arranged in a non-uniform manner and there can be and preferably are more locking slots 412 than ribs 402. In the illustrated embodiment, the locking slots 412 are arranged in a first set 432 that comprises three locking slots 412; a second set 434 that comprises three locking slots 412; and a pair of single slots 412 spaced 180 degrees opposite one another. The first and second sets 432, 434 are opposite one another (180 degrees one another). The central hub 325 is symmetric in nature as shown.

The second face 408 of the central hub 325 can represent a surface to which a fastening element 436 is coupled for securely coupling the central hub 325 to the canopy structure. More specifically, the fastening element 436 can be used to securely attach the central hub 325 to the first main canopy 310. As shown, the central hub 325 is disposed within the vent 325 of the first main canopy 310.

Any number of different types of fastening elements 436 can be used to securely attach the central hub 325 to the canopy structure. In the illustrated embodiment, the fastening element 436 is in the form of a pair of locking straps 438, 440. The first locking strap 438 has first and second ends 442, 444 that are attached to the first main canopy 310 and similarly, the second locking strap 440 has first and second ends 446, 448 that are attached to the first main canopy 310.

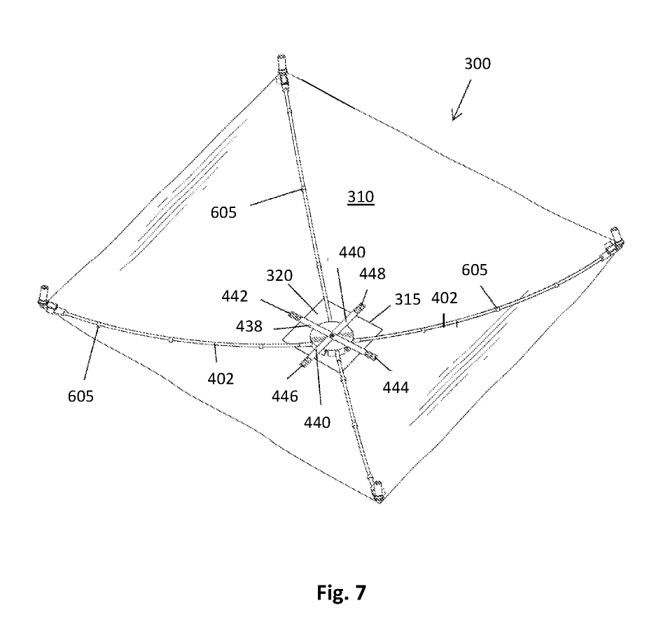

The straps 438, 440 are overlaid such that the two straps 438, 440 are centrally positioned and generally form a cross-hair shaped structure. The straps 438, 440 are secured to the hub 325 using a fastener 450 or the like (e.g., rivet, screw, nail, etc.). In the illustrated embodiment, the straps 438, 440 overlap in a central portion of the hub 325 and the fastener 450 passes through the overlapped portions of the straps 438, 440. The straps 438, 440 are thus attached to the second face 410 of the hub 325. The first and second ends 442, 444 of the strap 438 are attached to two opposing edges of the first main canopy 310 and more particularly, the ends 442, 444 are attached to two edges that define the vent 325 (see FIG. 7). As mentioned previously, the vent 325 has a square shape and thus, the two edges represent two sides of the square. Similarly, the first and second ends 446, 448 of the strap 440 are attached to two other opposing edges of the first main canopy 310 and more particularly, the ends 446, 448 are attached to two other edges that define the vent 315 (see FIG. 7). As mentioned previously, the vent 315 has a square shape and thus, the two edges represent the other two sides of the square.

It will be understood that the fastener 436 can be different than the straps 438, 440. For example, another type of mechanical attachment can be formed between the straps 438, 440 and the hub 325 or an adhesive or other type of bonding agent can be used to attach straps 438, 440 to the hub 325.

However, the fastener 436 should be of a type that allows for the movement of the central hub 325 in the manner described herein. In particular, forces are applied to the central hub 325 to cause inversion thereof as described herein.

The central hub 325 can be constructed so as to be a universal hub in that the inclusion of extra locking slots 412 allows for the use of different numbers of ribs 402. For example in the illustrated embodiment, only four ribs 402 are connected to the central hub 325 and therefore, there is a plurality of open locking slots 412. If the canopy has a different shape and instead is a hexagon or octagon, then the same central hub 325 can be used and simply, additional ribs are mounted within these additional locking slots of the central hub. In addition, it will be seen that when the fastener is in the form of a strap construction, the strap portions extend across an area of the central hub 325 and in fact can cover locking slots formed in the central hub 325.

The locking slots are thus arranged circumferentially about the central hub 325.

The central hub 325 can be formed of any number of different materials including plastics, wood, or other suitable materials. In the illustrated embodiment, the central hub 325 is formed of plastic and comprises a molded structure.

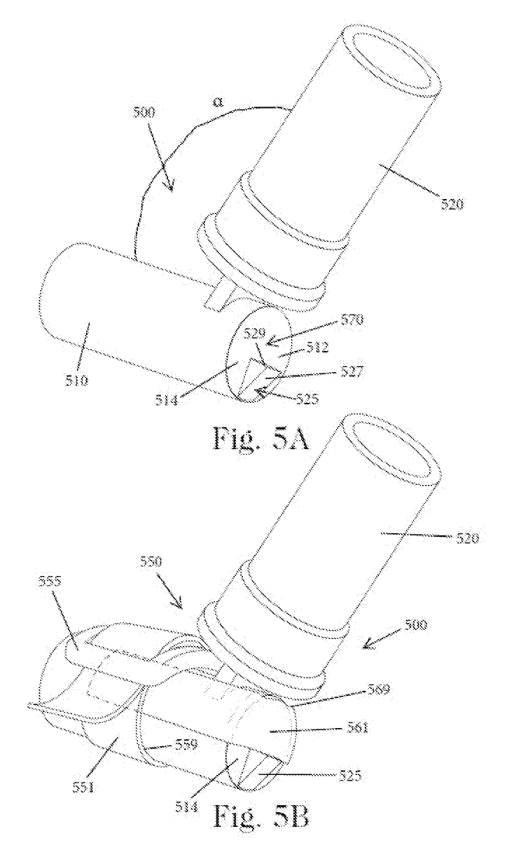

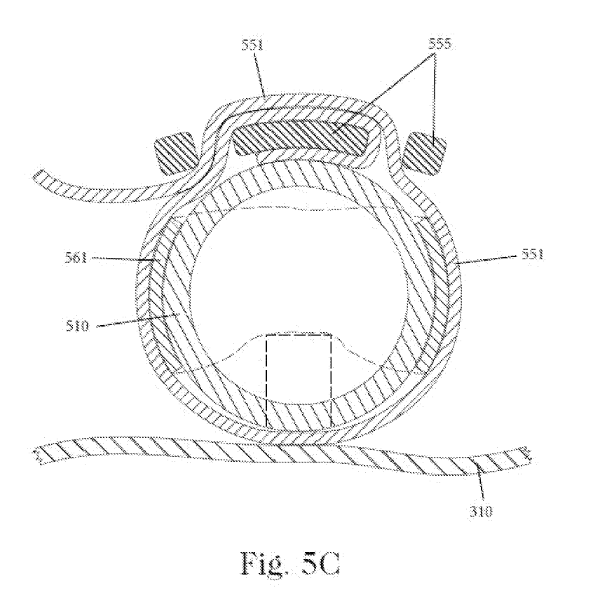

FIGS. 5A-C show a connector 500 that is intended to connect rib 402 to the first main canopy 310 and also to connect to one leg of the side wall assembly (805; see FIG. 8A). The connector 500 includes a first tubular portion 510 configured to receive the second end 405 of the rib 402 and a second tubular portion 520 that is configured to receive one end of the leg 805 of the side wall assembly 800. As shown, the first and second tubular portions 510, 520 are formed at an angle relative to one another. In one embodiment, the second end 405 of the rib 402 is retained within the hollow bore of the first tubular portion 510 by a friction fit and similarly, the leg of the side wall assembly is retained within the hollow bore of the second tubular portion 520 by a friction fit. However, it will be understood that any other means can be used to couple both the rib 402 and the leg of the side wall assembly to the connector 500. The connector 500 should be constructed such that the rib 402 and the leg of the side wall assembly can be removed from the connector 500 to allow disassembly of the product to allow storage and transportation thereof.

The dimensions of the first tubular portion 510 are less than the dimensions of the second tubular portion 520 since the dimensions of the rib 402 are less than the dimensions of the leg of the side wall assembly.

The first tubular portion 510 includes a closed end 512. The closed end 512 defines a planar outer surface 514. Along the planar outer surface 514, a protrusion 525 is formed. The protrusion 525 acts as a retention means for releasably retaining the first main canopy 310 to the connector 500. As illustrated, the protrusion 525 can be an angled, ramp like structure and in particular, can be generally triangular shaped. The protrusion 525 thus has an angled surface 527 and a vertical surface 529 that intersects the angled surface 527 and can be formed at a right angle to the planar surface 514.

A coupling member (connector) 550 is provided to attach the first main canopy 310 to the connector 500. The coupling member 550 is thus fixedly secured to the first main canopy (310) and configured to be detachably coupled to the connector 500, thereby securing the first main canopy (310) to the connector 500. In one embodiment, the coupling member 550 is in the form of a strap that is fixedly attached to the first main canopy (310). The strap 550 can be a nylon strap that is attached to the first main canopy (310).

Preferably, the coupling member (strap) 550 is adjustable in nature to allow a secure fit and attachment to be realized between the connector 500 and the first main canopy (310). For example, the strap 550 includes a buckle 555 which allows the length of the strap 550 to be adjusted.

As shown in FIGS. 5B and 5C, the strap 550 has two distinct portions, namely, a first strap portion 551 that is attached (e.g., stitched) to the first main canopy (310) and is coupled and releasably secured to the first tubular portion 510 and a second strap portion 561 that is also releasably secured to the first tubular portion 510 at a different location than the first strap portion 551 as described herein.

The first strap portion 551 can be in the form of an elongated strap that has first and second ends. The first strap portion 551 is fixedly attached to the first main canopy (310) at an intermediate location between the first and second ends. As mentioned, any number of suitable techniques can be used to attach the first strap portion 551 to the first main canopy (310) including but not limited to using stitching or the like or a mechanical fastener or adhesive/bonding agent. One end of the first strap portion 551 is coupled to the buckle 555 and the other end of the first strap portion 551 passes through a slot formed in the buckle 555 so as to allow the length of the first strap portion 551 to be adjusted. When coupled to the buckle 555, the first strap portion 551 defines a first loop 559 and the first tubular portion 510 is received within this first loop 559. The tightening of the first loop 559 about the first tubular portion 510 provides a means for coupling (attaching) the first main canopy (310) to the connector 500 and thereby provide a means for receiving the legs of the side wall assembly. The adjustability of the first loop 559 allows for different sized connectors 500 to be used with the coupling member 550. By tightening the first loop 559, a secure attachment is achieved between the connector 500 and the first main canopy 310 and conversely, by loosening the first loop 559, the connector 500 can be detached from the first main canopy 310.

The second strap portion 561 can be in the form of a fixed second loop 569 that is fixedly connected at the ends of the loop 569 to the first strap portion 551. For example, two opposite ends of the second strap portion 561 can be fixedly attached to the first strap portion 551 as by stitching or the like or using any other suitable techniques, such as those described herein.

The length of the second loop 569 is thus fixed and not adjustable.

As mentioned, the second loop 569 also provides a means for securely attaching and retaining the connector 500 in place relative to the first main canopy 310. More particularly, when the first strap portion 551 is attached to the first tubular portion 510 and is in a fairly taut condition, the second loop 569 is slid over the angled protrusion 525 (which acts as a cam) and once the second loop 569 clears the protrusion, the strap section 560 drops into place in a space 570 formed between the protrusion 525 and the second tubular portion 520. The second loop 569 is maintained within this space 570 in the taut condition (by an interference fit), thereby detachably yet securely attaching the first main canopy 310 to the connector 500.

It will be appreciated that as the second loop 569 moves along the angled protrusion 520 in an upward manner away from the peripheral edge of the planar outer surface 514, it becomes harder to push the second loop 569 due to the taut nature thereof. As mentioned above, the strap 550 can be adjusted using the buckle 555.

The adjustability of the strap 550 allows for the connector 500 to be used with different sized and different shaped canopies 310.

The connector 500 and coupling member 555 thus provide a manner for detachably securing the canopy 310 to frame structure. FIG. 7 shows the first main canopy 310 attached to the frame structure. FIG. 7 also shows the bottom of the canopy structure 300 in an in-use state in which the first main canopy 310 is under tension.

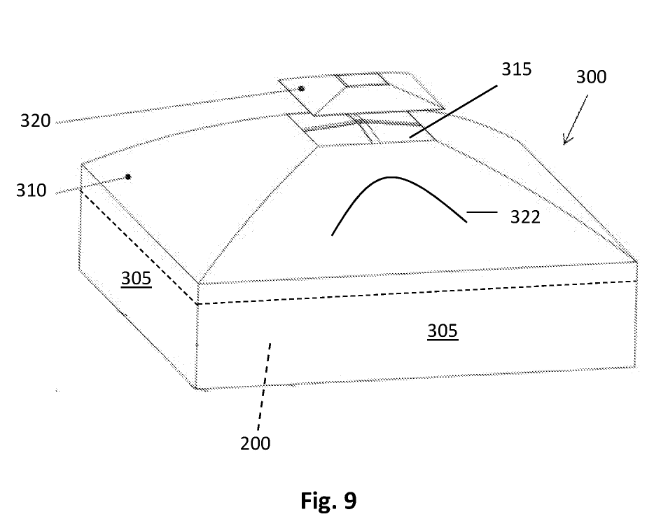

In one or more embodiments, the canopy structure 300 is formed such that in the fully opened position (FIGS. 7 and 9), the angle between the canopy 310 and each leg of the side wall assembly is between about 108 degrees and about 118 degrees. In one embodiment, the angle is between about 111 degrees and 115 degrees, and more particular, between about 112 degrees and 114 degrees and more particularly, the angle can be about 113 degrees in one embodiment. It will also be understood, as shown in FIG. 5A, that the angle a between the tubular portion 510 and the tubular portion 520 is similar and in particular, the angle can be between about 108 degrees and about 118 degrees. In one embodiment, the angle is between about 111 degrees and 115 degrees, and more particular, between about 112 degrees and 114 degrees and more particularly, the angle can be about 113 degrees in one embodiment.

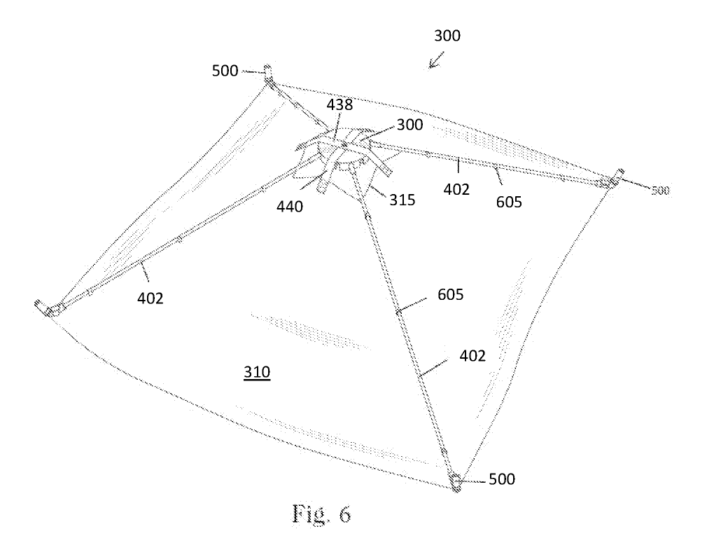

FIG. 6 shows the canopy structure 300 in a knock-down state in which the first main canopy 310 is not under tension. As shown in FIG. 6, the ribs 402 can be held in place along the first main canopy 310 by one or more retainers 605. The retainers 605 can have any number of different types of structures including, but not limited to, ties, hook and loop fasteners, loops, etc.

With continued reference to FIG. 6, the canopy structure 300 is configured such that in the initial state, the ribs 402 extend in a substantially linear manner between the central hub 325 and the connectors 500 in each corner of the first main canopy 310. As a result, the central hub 325 and the inner section of the first main canopy 310 are raised relative to the ground surface when the canopy structure 300 is placed on the ground surface with its top surface on the ground surface.

The canopy structure 300 is constructed to allow the user to easily cause the canopy structure 300 to assume the in-use state (FIG. 7) from the initial (relaxed) state shown in FIG. 6. More specifically, to cause such change in states, the user applies a force to the central hub 325 in a downward direction (toward the ground surface on which the canopy structure rests) to cause an inversion action resulting in the canopy structure 300 assuming the in-use state shown in FIG. 7. More specifically, the user can place his or her foot on top of the central hub 325 and push down toward the ground surface, the ribs 402 flex inward and assume curved positions as shown in FIG. 7. Under tension, the ribs 402 assume the curved positions. In other words, since the two ends of each rib 402 is fixedly attached to a corresponding structure (central hub 325 and the connector 500), the movement of the central hub 325, while the connector 500 remain relatively in the same positions, causes the inversion of the canopy and the bending of the ribs 402.

Once the canopy structure 300 assumes the in-use position shown in FIG. 7, the user can then attach the legs of the side wall assembly to the frame structure and more particularly, to the connectors 500. For example, the legs of the side wall assembly can be implanted into the ground or otherwise positioned in a self-standing manner relative to the ground surface and then the user can position the canopy structure such that the legs of the side wall assembly can be slidingly inserted into the first tubular portion 510 of the connector 500, thereby fixing the canopy and frame structure to the legs of the side wall assembly.

It will also be understood that the converse action is likewise true in that after use of the canopy structure 300, the user first disassembles the canopy and frame structure from the legs of the side wall assembly. The detached canopy and frame structure are then placed on the ground surface with the top face of the canopy structure facing upward (i.e., as in the position of FIG. 3A and opposite to that shown in FIG. 7). When placed on the ground surface in this position, the central hub 325 is elevated relative to the ground surface on which the connectors 500 rest. In this canopy orientation, the ribs 402 are still under tension and exhibit curvature along their lengths. The user then applies a downward force (as by using foot action) to cause inversion of the canopy and frame structure as a result of the release of the tension stored in the ribs 402. The detached canopy and frame structure then assumes the relaxed state that is suitable for folding up and storing and/or transporting the canopy and frame structure.

Unlike conventional canopy structures which require assembly of the components of the frame and typically require the user to use his or her hands to assemble the canopy structure and/or to move the canopy structure between a collapsed (relaxed) state and an in-use state, the present invention is configured such that the user can user his or her foot to alter the state of the canopy structure (i.e., move between the collapsed and in-use states).

In addition and according to one other aspect of the present invention, the central hub is constructed such that an angle of the central hub, ribs and the canopy structure is carefully controlled such that after deploying the canopy structure to the in-use position, the entire collapsible shelter will fit for a four (4) foot square or a four (4) foot hexagon shaped playground. Without carefully control of the angle, the deployed collapsible shelter will not fit into a four (4) foot playground. Thus, the angle is important to control and tailor the overall footprint of the collapsible shelter.

It will also be appreciated that due to the design of the main components of the collapsible shelter and in particular, the dome-shaped construction, a user can easily open and close the collapsible structure within seconds. As discussed herein, to open the canopy structure 300, the loose cloth of the main canopy 310 is spread out on a ground surface and the canopy structure 300 assumes a non-tensioned convex shape (FIG. 6) and by pressing the central hub, the collapsible shelter inverts and assumes a tensioned concave shape (FIG. 7). To close the collapsible canopy structure 300, the tensioned concave shaped structure is turned over so as to position the tensioned canopy structure (having a convex shape) on the ground surface with the central hub being elevated from the ground surface and then the user presses the central hub to collapse the structure back the non-tensioned convex shape of FIG. 6.

In yet another embodiment, cross supports can be provided for increasing the robustness and rigidity of the frame structure that supports the canopy structure. In particular, between a set of legs of the side wall assembly, a cross member can be provided to be releasably attached at its two ends to the set of legs. More specifically, each leg of the side wall assembly can include a side connector which receives one end of the cross member. Any number of different types of side connectors can be used so long as the side connector is configured to receive and releasably couple to (e.g., interlock with) the cross member. It will also be appreciated that the cross member can be an elongated collapsible structure, such as a telescoping cross member structure.

It will be understood that other canopy structures can be used and the present invention is not limited to the use of canopy structure 300.

Side Wall Assembly

FIG. 8A shows an exemplary side wall assembly 800 in accordance with one or embodiments. The side wall assembly 800 can comprise one more legs 805, which connect the side wall assembly 800 to the canopy structure 300 (e.g., via connector 500, see FIGS. 5-7) and to the raised garden base 200 (e.g., via bore 245, see FIG. 2). In the embodiment of FIG. 8A, there are four sides to the conservatory 100 and therefore, up to four side walls 810 can be installed. However, it will be appreciated that the conservatory 100 can have three, five, six, or more legs 805, and thus additional side walls 810 as shown in FIG. 8B. As explained herein, the second ends 835 of the poles 805 can be in the form of stakes; however, when the second ends 835 mate with bore 245 of the base 200, they can be simply tubular ends and not stakes. Side walls 810 can be similar or identical to side walls 305. The side walls 810 depend downward from the canopy structure 300.

In one or more embodiments and as shown in FIG. 8A and FIG. 8B, the side wall assembly 800 features side walls 810, where each side wall 810 is located between a pair of legs 805. The side walls 810 are selected to have a length that results in the top edge being connected to the canopy structure 300 and the bottom edge of each side wall 810 being either connected with or in close proximity to the raised base 200 or arranged such that the bottom edge is in contact or adjacent the ground with the bottom portion of the side wall covering the outer surface of the side wall.

Any number of techniques can be used to attach the side walls 810 to each other and to respective sides (edges) of the canopy structure 300 and in particular, the technique can be of a type that allows the side walls 810 to be detachably attached to the canopy structure 300 so as to allow the side walls to be an optional feature or to be selectively removed or selectively attached. For example, as shown in FIG. 8C, the fasteners 825, such as hoop and loop pieces, snaps, buttons, zippers, etc., can be used to attach the ends of each side wall 810 to an adjacent side wall 810. Similarly, as shown in FIG. 8D the fasteners 825 can be used to attach the top ends of the side walls 810 to respective sides (side panel 305) of the canopy structure 300 at select locations thereof (i.e., at bottom edges of the sides of the canopy structure 300). Thus, for instance, hook and loop fasteners can be placed at select locations across the side of the canopy structure and similarly, hook and loop fasteners can be placed at select locations along the backside of the side wall, as discussed in greater detail below. The side walls 810 can be selectively removable to provide users with easy access to the inside of the conservatory 100. Side walls 810 can be removed to allow the entrance or exit of the butterflies during migration seasons. As discussed herein, the access of the butterflies can instead be through the openable windows in the canopy structure 300 as opposed to through openings in the side walls 810.

Once again, it will be appreciated that the conservatories shown in FIGS. 8A and 8B can be formed of the same parts but have different configurations.

In one more embodiments, the side walls 810 can be made of a plastic material, such as PVC or other materials. In one or more other embodiments, the side walls 810 can be made of a mesh material. In this embodiment, the side walls 810 allow air to flow through the conservatory 100, but also provides protection for the plants and caterpillars/butterflies from precipitation and from predators and parasites. The mesh side walls 810 also offer a suitable surface for chrysalis formation for the butterflies. In one or more implementations, the mesh side walls 810 can be a dark colored mesh (e.g., black mesh), which can reduce the harsh sunlight, thereby preventing desiccation of butterfly eggs and caterpillars. The reduced light also keeps adult butterflies calmer, thereby reducing wing damage. The dark colored walls also make it easier for users to see the butterfly eggs against the leaves of the host plants. The side walls 810 also provide protection for plants from frost during spring and fall months. In one or more implementations, one or more of the side walls 810 can be printed to display a landscape or nature-themed scene, or the like.

It will also be appreciated that one or more of the side walls 810 can include a door 815 (FIG. 8B) and/or one or more windows 820. In FIG. 8A, the door is indicated at 303. The door 815 and/or windows 820 can be integral to the side wall 810 can be selectively opened by a user via one or more fasteners mechanisms such as a zipper. In embodiments in which the conservatory 100 is used for housing plants and/or caterpillars/butterflies, the windows 820 can be selectively left open to invite passing butterflies into the conservatory 100 lay eggs or to allow butterflies to exit the conservatory 100. The windows 820 also allow for easy access for users to water plants located within the conservatory 100, or to allow spectators to view the plants and/or insects within the conservatory 100. The windows 820 can also be selectively closed to protect the conservatory from the elements (e.g., wind, rain), and to protect the caterpillars and butterflies from predators and parasites.

In another embodiment, the windows 820 can be formed of a different material than the material that forms the surrounding side wall sections 810. For example, the side wall can be formed primarily of a mesh material with the exception that the windows 820 can be a transparent PVC material to allow a user to easily see into and out of the structure. The window 820 can thus be formed of a non-mesh material.

In one or more implementations, the door 815 can feature a double zipper to allow for opening of the door 815 from inside and outside of the conservatory 100. In certain embodiments, the door 815 can also be a double wall being composed of two separate layers (e.g., mesh). The double wall allows a user to enter and/or exit the conservatory 100 while preventing the escape of the butterflies from the conservatory.

In one or more embodiments, side wall fasteners 825 can be included and used to further attach the sides of the side wall 810 to legs (poles) 805. These fasteners 825 can be in the form of loops, ties, etc. (hook and loop material) that allow the sides of the side wall 810 to be connected to the pole 805 at various locations. The fasteners 825 can be integrally attached to the sides of the side wall 810 or they can pass through slits formed in the side walls 810.

Now returning to FIG. 8A, four exemplary legs 805 are shown. Each leg 805 is an elongated structure having a first end 830 and an opposing second end 835. In one or more implementations, the leg 805 can be a stationary leg that is flexible in order to help withstand wind and other elements. In at least one implementation, the leg 805 can be of a telescoping type in that a length of the leg 805 can be altered and this allows the leg 805 to be moved between a collapsed state (for storage) and an extended state (for use). As is known, the telescoping leg 805 can be formed of a plurality of leg sections that nest with each other in the collapsed state and have a locking mechanism associated therewith for locking the leg sections in the extended state. Any number of suitable locking mechanisms can be used for locking the telescoping leg sections in place including clamp type locking mechanism.

The legs 805 shown in FIG. 8A and FIG. 8B can be of the type that is intended to be inserted into the bores 245 of the raised garden base 200. Locking mechanism can be used to lock the second ends 835 in place in the base 200 or by a friction fit or keyed fit or the like. However, in one or more embodiments, the legs 805 can also be inserted directly into a ground surface. For example, as shown in FIG. 8A, the leg 805 can be of a stake type with the second end 835 being a sharp pointed end configured to be implanted into the ground surface. Suitable ground surfaces for a stake type leg include but are not limited to a dirt surface, sand, grass covered dirt area, etc.

### CONFIGURATIONS

### Cold Frame Configuration

FIG. 9 shows a cold frame configuration in which the canopy structure 300 is directly connected to the raised base 200 in accordance with at least one embodiment. As mentioned herein, the cold frame configuration is used during the cool months to initially start plants that attract the butterflies and provide a food source. The canopy structure 300 is directly attached to the raised garden base 200 (which is shown behind the panels 305). In particular, the frame of the canopy structure is coupled to the raised garden base 200 as by inserting small poles between the canopy structure and the raised garden base 200 or by inserting the second tubular portion 520 of the canopy structure into the bore of the connectors of the raised garden base 200. In this way, the canopy structure 300 is directly connected to the raised garden frame and is in a lowered position where the apex of the roof is low to the ground over the raised garden base 200. In other words, the long poles shown in FIG. 8A for the raised configuration are not used but instead, shorter poles are used or canopy 300 itself is directly attached. In any event, the structure 300 is securely coupled to the base 200. The apex of the canopy can be located only a number of feet above the ground and in close proximity to the top of the base 200.

Since the seedlings are low to the ground in their initial planted state, the lowered canopy structure 300 is at sufficient height to allow growth of the seedlings, while offering the necessary protection including shielding the seedlings from animals and also the elements, such as heavy rain, wind, frost, etc. In addition, heat is trapped underneath the canopy structure 300 so as to provide heat to the seedlings and provide an optimal growing environment. The windows 322 formed in the canopy structure 300 allow for the user to directly access the seedling and this allows the user to easily water and otherwise care for the plants. The windows 322 can also be left at least partially open to allow for rain and air to enter. The vent 315 at the apex of the canopy structure 300 allows for air to enter at all times, while effectively preventing water ingress.

The transparency and material selection of the material forming the canopy structure 300 allows for sunlight to pass through the canopy structure 300 and the overall structure of the cold frame allows most plants to get an early start in the planting season.

Once the cool weather passes and the seedling mature into larger plants, the butterfly conservatory is then transitioned from the cold frame configuration to the upright raised configuration.

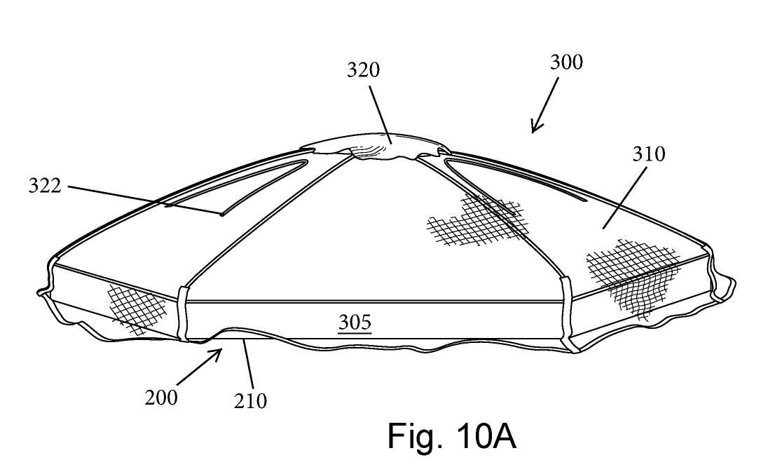

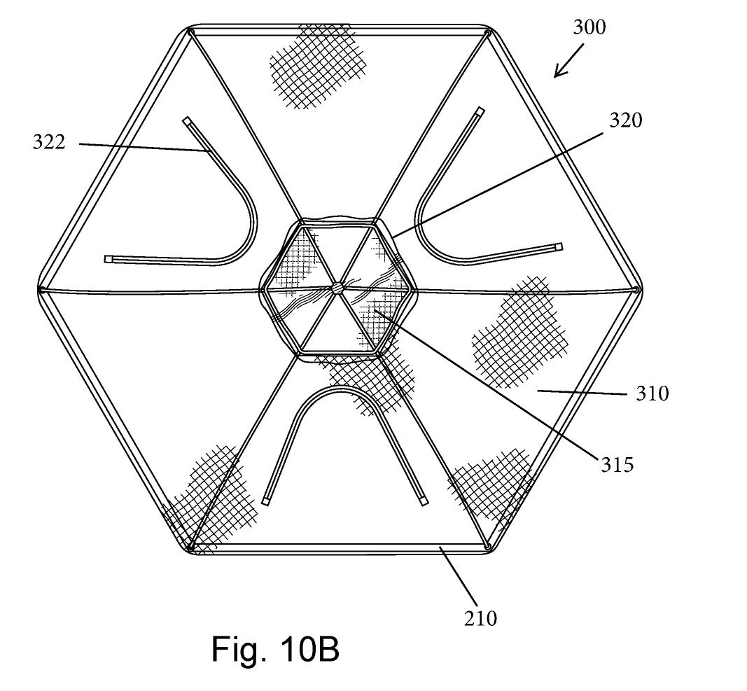

FIGS. 10A and 10B show another embodiment cold frame configuration in which the canopy structure 300 is directly connected to the raised base 200. In particular, FIG. 10A shows a side view of the canopy structure and the raised garden base in the cold frame configuration and FIG. 10B is a top view of the same configuration. In this embodiment, the canopy structure 300 and raised base are in a hexagonal shape. While the cold frame configuration has been shown in a generally square configuration (FIG. 9) and a generally hexagonal configuration (FIGS. 10A-B), it should be understood that the cold frame configuration (including the shape of the canopy structure and the raised base) can be formed into any number of different shapes.

As shown in FIG. 9 and FIG. 10A, the side walls 305 of the canopy 300 can be laid over the outer surfaces of the rails of the base structure 200.

### Raised Frame Configuration

Returning now to FIG. 8B, a raised frame configuration is shown in which the canopy structure 300 is raised relative to the raised garden base 200 by insertion of the poles 805 into the raised garden base 200 which serve to raise the canopy structure 300. After insertion of the poles 805, the side walls 810 are then added by detachably coupling each side wall 810 to one respective side wall of the canopy structure 300 and positioning the bottom of the side wall 810 proximate or adjacent one section of the raised garden base 200 and the bottom can be detachably attached to base 200 using fasteners, or the like. In at least one implementation, the raised frame configuration does not include the raised garden base 200, and thus the poles 805 are inserted directly into the ground. The raising of the canopy structure into the raised frame configuration results in the butterfly conservatory being assembled and ready for use. In the raised configuration, the apex of the canopy 300 is at set a greater distance from the base structure 200 as shown than the cold frame configuration.

The mesh material of the side walls 820 allows for air to freely flow into the interior of the butterfly conservatory. The door allows for easy access into the interior. As mentioned herein, the side walls 810 serve to provide a protective environment for butterflies and caterpillars that turn into the butterflies. Once the canopy structure 300 is raised to allow for further growth of the plants that become the food source for the hatched caterpillars, select access can be provided into the interior of the butterfly conservatory as by opening one or more window formed in the canopy structure 300. The openings of the windows permit butterflies that are migrating home to land and enter the interior of the conservatory via the windows. The butterflies enter to access the grown plants which attract the butterflies. For example, and as mentioned herein, monarch butterflies eat milkweed and thus, if milkweed plants are grown in the conservatory, the scent of the milkweed will attract monarch butterflies. As mentioned herein, the scent beacon formed at the apex of the vent 315 of the canopy structure 300 serves to funnel the scent of the milkweed to the exterior of the conservatory for attracting the monarch butterflies.

Once the butterflies enter the interior of the conservatory, the butterflies encounter favorable conditions for laying eggs to begin the life cycle again. As mentioned herein, the eggs are laid on the plants that provide the food source for the caterpillars. The butterflies thus lay eggs on the leaves of the plants being grown in the conservatory. These eggs are protected once again from harsh conditions, such as heavy rain and/or rain or predators, etc., by the enclosed nature of the conservatory. The eggs laid on the leaves hatch into caterpillars. The hatched caterpillars feast on the leaves of the plant and continue to grow until it is time for the next stage of the life cycle to begin.

More specifically one day, the caterpillar stops eating, hangs upside down from a suitable support structure, such as a leaf, and spins itself a silky cocoon or molts into a shiny chrysalis. Within its protective casing, the caterpillar radically transforms its body, eventually emerging as a butterfly or moth. As mentioned herein, the mesh vent 315 provides an optimal material upon which the chrysalis can be formed. This mesh vent 315 is located at the apex of the canopy structure 300 and allows the caterpillar to easily hang itself to allow for formation of the chrysalis.

The additional mesh material that is used for the side walls, etc., can also provide an area to which a chrysalis can be formed.

After chrysalis formation, the conservatory provides a safe environment for the evolution of the chrysalis into a butterfly. Once the butterfly is released from the chrysalis, the conservatory provides a protected initial environment for the new butterfly in that the raised garden includes plants that provide a food source for the butterfly. Once the butterfly becomes stronger, the butterfly can be released by opening up one or more windows in the canopy structure 300 or as described below, one or more of the side walls can be removed.

The present invention thus provides a butterfly conservatory kit that includes multiple features, described herein, that promote butterfly migration and multiplication by being configured to first provide a cold frame configuration to initially grow the butterfly's food source and a raised frame configuration that can act as a greenhouse allowing for additional growth of the food source plants and defining a closed structure into which one or more persons can enter into the interior to learn from and interact with the butterflies. The raised frame configuration can also act as barrier to animals to prevent damage to the plants and/or butterflies. The modular aspect of the conservatory allows for customization of different features, such as side wall configurations, colors, graphics, locations of windows, door(s), etc.

### Autumn Migration

To promote the annual fall migration of the butterflies, one or more of the side walls 810 can be removed as by detaching the respective side walls from the side wall portion of the canopy structure 300, thereby forming a more open structure for the butterflies to depart and set off on their journey.

It will be apparent from the foregoing that while particular forms of the invention have been illustrated and described, various modifications can be made without departing from the spirit and scope of the present invention. Accordingly, it is not intended that the invention be limited, except as by the appended claims.

It is to be understood that like numerals in the drawings represent like elements through the several figures, and that not all components and/or steps described and illustrated with reference to the figures are required for all embodiments or arrangements.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components, and/or groups thereof.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

The subject matter described above is provided by way of illustration only and should not be construed as limiting. Various modifications and changes can be made to the subject matter described herein without following the example embodiments and applications illustrated and described, and without departing from the true spirit and scope of the present disclosure, which is set forth in the following claims.

What is claimed is:

1. A reconfigurable butterfly conservatory comprising:
a raised garden base that defines an inner garden area, the raised garden base including holes for receiving complementary upright poles;
a canopy structure that includes connectors for receiving the complementary upright poles, wherein the canopy structure includes an air vent and one or more first windows that can be opened and closed, wherein the air vent includes a mesh layer that promotes chrysalis formation; and
a plurality of side walls that are configured for coupling to at least the canopy roof structure for suspending the plurality of side walls such that bottom edges of the side walls are positioned adjacent to the raised garden base to define a hollow interior space that includes the inner garden area, wherein at least one side wall is formed at least substantially of a mesh material.

2. The reconfigurable butterfly conservatory of claim 1, wherein the raised garden base includes a plurality of supports that are interconnected by a plurality of connectors to define the inner garden area that is located internal to the interconnected supports.

3. The reconfigurable butterfly conservatory of claim 2, wherein one or more connectors includes the holes for receiving first ends of the upright poles.

4. The reconfigurable butterfly conservatory of claim 2, wherein the supports comprises wood planks.

5. The reconfigurable butterfly conservatory of claim 1, wherein the canopy structure includes a flexible first roof portion that is supported by a foldable frame, the one or more first windows being formed in the first roof portion.

6. The reconfigurable butterfly conservatory of claim 5, wherein the first roof portion is formed of a transparent material.

7. The reconfigurable butterfly conservatory of claim 5, wherein the first roof portion is formed of a flexible PVC material.

8. The reconfigurable butterfly conservatory of claim 5, wherein each first window is openable and closeable.

9. The reconfigurable butterfly conservatory of claim 8, wherein each window is defined by a slit formed in the first roof portion and a fastener that is configured to close the slit when the window is in a closed position.

10. The reconfigurable butterfly conservatory of claim 9, wherein the fastener comprises a zipper.

11. The reconfigurable butterfly conservatory of claim 5, wherein the first roof portion includes a plurality of side wall portions that include bottom edges that include first fasteners and the plurality of side walls include top edges that include second fasteners that are configured to mate with the first fasteners to detachably connect the side walls to the canopy structure.

12. The reconfigurable butterfly conservatory of claim 11, wherein the first and second fasteners are selected from the group consisting of: hook and loop fasteners, snaps, zippers, and buttons.

13. The reconfigurable butterfly conservatory of claim 5, wherein the air vent includes an upper vent panel that is attached to the first roof panel at select locations and covers a vent opening formed in the first roof panel, the mesh layer also covering the vent opening.

14. The reconfigurable butterfly conservatory of claim 13, wherein the mesh layer is attached to the first roof panel so as to cover the vent opening formed in the first roof panel.

15. The reconfigurable butterfly conservatory of claim 1, wherein the air vent is formed at an apex of the canopy structure.

16. The reconfigurable butterfly conservatory of claim 1, wherein at least one of the side walls includes an openable and closeable door.

17. The reconfigurable butterfly conservatory of claim 1, wherein at least one of the side walls includes a window.

18. The reconfigurable butterfly conservatory of claim 17, wherein the window is formed of a first material that is different than a second material that forms a surrounding portion of the side wall.

19. The reconfigurable butterfly conservatory of claim 18, wherein the first material is a non-mesh material and the second material is mesh.

20. The reconfigurable butterfly conservatory of claim 18, wherein the first material is a transparent polymeric material and the second material is mesh.

21. The reconfigurable butterfly conservatory of claim 1, wherein the butterfly conservatory is positionable between: a cold frame configuration in which the canopy structure is directly attached to the raised garden base and the plurality of side walls are not used and a raised canopy configuration in which poles suspend and position the canopy structure above the raised garden base.

22. The reconfigurable butterfly conservatory of claim 21, wherein in the cold frame configuration, side wall portions of the canopy structure drape over the raised garden base.

23. The reconfigurable butterfly conservatory of claim 1, wherein the plurality of side walls includes a first side wall that includes a first vertical edge at one side thereof and a second side wall that includes a second vertical edge at one side thereof, wherein the first vertical edge includes a first vertical fastener and the second vertical edge includes a second vertical fastener that mates with the first vertical fastener to close off and defined a continuous side wall structure.

24. The reconfigurable butterfly conservatory of claim 23, wherein the first and second vertical fasteners comprise hook and loop fasteners.

25. A reconfigurable butterfly conservatory kit comprising:
a raised garden base that defines an inner garden area, the raised garden base including holes for receiving complementary upright poles;
a canopy structure that includes connectors for receiving the complementary upright poles, wherein the canopy structure includes an air vent and one or more first windows that can be opened and closed, wherein the air vent includes a mesh layer that promotes chrysalis formation; and
a plurality of side walls that are configured for coupling to at least the canopy roof structure for suspending the plurality of side walls such that bottom edges of the side walls are positioned adjacent to the raised garden base to define a hollow interior space that includes the inner garden area, wherein at least one side wall is formed at least substantially of a mesh material;
wherein the butterfly conservatory kit can be configured as: (1) a cold frame configuration in which the canopy structure is directly attached to the raised garden base for allowing for planting and initial growth of seedlings and the plurality of side walls are not used and (2) a raised canopy configuration in which poles suspend and position the canopy structure above the raised garden base and the plurality of side walls define and enclose a hollow interior space.
